(19) United States
(12) Patent Application Publication
(10) Pub. No.: US 2018/0092682 A1
(43) Pub. Date: Apr. 5, 2018

(54) SPIRAL ELECTRODE FOR NEUROMODULATION THERAPY

(71) Applicant: Medtronic Ardian Luxembourg S.a.r.l., Luxembourg (LU)

(72) Inventors: Jason Lawinger, Minnesota, MN (US); Alan Connors, Minneapolis, MN (US); Jeffrey Vogel, Brooklyn Park, MN (US)

(21) Appl. No.: 15/286,339

(22) Filed: Oct. 5, 2016

(51) Int. Cl. A61B 18/02; A61B 18/14; A61M 25/09; A61F 7/12; A61B 18/06; A61N 1/30

(52) U.S. Cl. CPC A61B 18/02; A61B 18/14; A61M 25/09; A61B 2018/00077; A61B 18/06; A61N 1/303; A61B 2218/002; A61F 7/12

(57) ABSTRACT

Catheters having spiral electrodes and methods for their use in neuromodulation therapy. A therapeutic assembly is disposed at a distal portion of a neuromodulation catheter and is adapted to be located at a target location within a target blood vessel of a human patient. The therapeutic assembly can include at least two shape-memory metallic elements that extend in parallel along the distal portion, each metallic element having an exposed surface to serve as an electrode. The shape memory of the metallic elements transforms the therapeutic assembly between a low-profile delivery configuration and a deployed radially-expanded spiral configuration. A dielectric material attaches the metallic elements to each other while maintaining physical separation and electrical isolation from each other along at least the distal portion.

### TECHNICAL FIELD

The present technology is related to neuromodulation. In particular, various embodiments of the present technology are related to devices having generally spiral-shaped metallic elements separated by a dielectric material for intravascular renal neuromodulation and associated methods.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is a partially schematic illustration of a neuromodulation system configured in accordance with an embodiment of the present technology.

FIGS. 2A and 2B are partially schematic side views of the neuromodulation system in a first state and a second state, respectively, positioned within a blood vessel of a human patient in accordance with an embodiment of the present technology.

FIG. 2C is a cross-sectional view of the neuromodulation system taken along line 2C-2C in FIG. 2B.

FIG. 2D is an enlarged detail view of a portion of the neuromodulation system shown in FIG. 2B.

FIG. 3A is a partially schematic side view of another embodiment of a neuromodulation system in an expanded state and positioned within a blood vessel of a human patient.

FIG. 3B is a cross-sectional view of the neuromodulation system taken along line 3B-3B in FIG. 3A.

FIG. 3C is an enlarged detail view of a portion of the neuromodulation system shown in FIG. 3A.

FIG. 4A is a partially schematic side view of yet another embodiment of a neuromodulation system in an expanded state and positioned within a blood vessel of a human patient.

FIG. 4B is a cross-sectional view of the neuromodulation system taken along line 4B-4B in FIG. 4A.

FIG. 4C is an enlarged detail view of a distal portion of the neuromodulation system shown in FIG. 4A.

FIG. 5 illustrates modulating renal nerves and/or evaluating the neuromodulation therapy with the system of FIG. 1 in accordance with an embodiment of the present technology.

FIG. 6 is a conceptual illustration of the sympathetic nervous system (SNS) and how the brain communicates with the body via the SNS.

FIG. 7 is an enlarged anatomic view of nerves innervating a left kidney to form the renal plexus surrounding the left renal artery.

FIGS. 8 and 9 are anatomic and conceptual views, respectively, of a human body depicting neural efferent and afferent communication between the brain and kidneys.

FIGS. 10 and 11 are anatomic views of the arterial vasculature and venous vasculature, respectively, of a human.

### DETAILED DESCRIPTION

The present technology is directed to apparatuses and methods for achieving electrically- and/or thermally-induced renal neuromodulation (i.e., rendering neural fibers that innervate the kidney inert, inactive or otherwise completely or partially reduced in function) by percutaneous transluminal intravascular access. In particular, embodiments of the present technology relate to a treatment device (e.g., treatment catheter) having a therapeutic assembly with shape-memory metallic elements separated transversely by a dielectric material. The metallic elements together cause a distal portion of the therapeutic assembly to tend towards a pre-formed, generally spiral shape. After deployment in a target blood vessel of a human patient, a distal portion of the assembly is transformable between a delivery state having a low-profile that is configured to pass through the vasculature and a deployed state having a radially expanded shape (e.g., generally spiral/helical or coil) in which the shape-memory metallic elements maintain the assembly in stable apposition with an inner wall of the target blood vessel (e.g., renal artery).

The system can also include an energy source or energy generator external to the patient in electrical communication with the metallic elements of the therapeutic assembly. In operation, the metallic elements are advanced to a target blood vessel, such as the renal artery, along a percutaneous transluminal path (e.g., a femoral artery puncture, an iliac artery and the aorta, a radial artery, or another suitable intravascular path), and then energy is delivered to the wall of the target blood vessel via the metallic elements. Suitable energy modalities include, for example, electrical energy, radio frequency (RF) energy, pulsed electrical energy, or thermal energy. The treatment device carrying the metallic elements can be configured such that the metallic elements are in constant apposition with the interior wall of the target blood vessel when in the deployed state (e.g., radially expanded to have a spiral/helical shape). The pre-formed spiral/helical shape of the deployed portion allows blood to flow through the assembly during therapy, which is expected to help cool the therapy assembly to prevent clot formation that may result in occlusion of the blood vessel during activation of the metallic elements. The spiral/helical shape also enhances the apposition of the metallic elements with the inner wall of target blood vessels and makes the therapeutic assembly adaptable to a range of vessel diameters. The largest diameter vessel in the range is at least slightly smaller than the free or un-constrained diameter of the pre-formed spiral/helical shape in order to provide and maintain adequate contact between the metallic elements and the vessel wall.

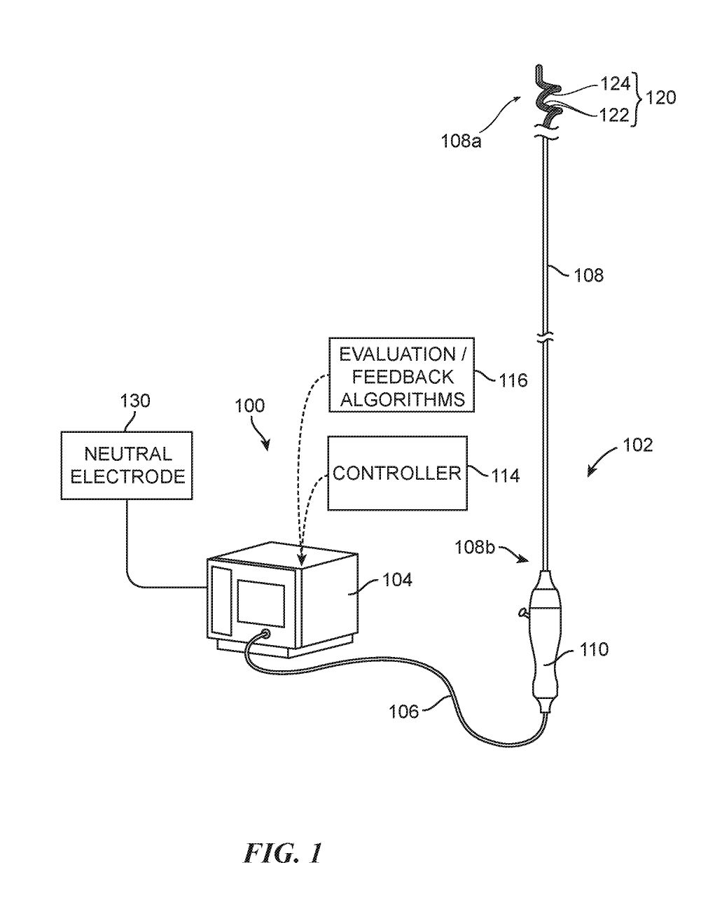

Specific details of several embodiments of the present technology are described herein with reference to FIGS. 1-11. Although many of the embodiments are described with respect to devices, systems, and methods for intravascular renal neuromodulation, other applications and other embodiments in addition to those described herein are within the scope of the present technology. For example, at least some embodiments of the present technology may be useful for intraluminal neuromodulation, extravascular neuromodulation, non-renal neuromodulation, and/or use in therapies other than neuromodulation. It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

As used herein, the terms “distal” and “proximal” define a position or direction with respect to a clinician or a clinician's control device (e.g., a handle of a neuromodulation catheter). The terms, “distal” and “distally” refer to a position distant from or in a direction away from a clinician or a clinician's control device along the length of device. The terms “proximal” and “proximally” refer to a position near or in a direction toward a clinician or a clinician's control device along the length of device. The headings provided herein are for convenience only and should not be construed as limiting the subject matter disclosed.

### Selected Examples of Neuromodulation Systems

FIG. 1 is a partially schematic illustration of a neuromodulation system 100 configured in accordance with still another embodiment of the present technology. The system 100 includes a neuromodulation catheter 102, a console 104, and a cable 106 extending therebetween. Cable 106 may provide a permanent connection between catheter 102 and console 104, or cable 106 may be disconnectable (e.g. to permit the use of console 104 with different catheters). The neuromodulation catheter 102 can include an elongated shaft 108 having a proximal portion 108b, a distal portion 108a, a handle 110 operably connected to the shaft 108 at the proximal portion 108b, and a neuromodulation assembly 120 operably connected to and/or comprising at least a part of the distal portion 108a. The diameter of shaft 108 and the neuromodulation assembly 120 can be 2, 3, 4, 5, 6, or 7 French or another suitable size. The neuromodulation assembly 120 can include two or more metallic elements 122 that extend longitudinally along at least a portion of the length of the neuromodulation assembly 120 and a dielectric material 124 between the metallic elements 122. The metallic elements 122 can be elongated electrodes that extend longitudinally along the neuromodulation assembly 120 and are configured to apply electrical stimuli (e.g., RF energy) to target sites at or proximate to vessels within a patient, to temporarily stun nerves, to deliver neuromodulation energy to target sites, and/or to detect vessel impedance. In various embodiments, certain metallic elements 122 can be dedicated to applying stimuli and/or detecting impedance, and the neuromodulation assembly 120 can include other types of therapeutic elements that provide neuromodulation therapy using various modalities, such cryotherapeutic cooling, ultrasound energy, etc. The dielectric material 124 can be an elongated element that extends along at least a portion of the length of the neuromodulation assembly 120 and separates the metallic elements 122 from each other along at least a portion of the length of the metallic elements 122.

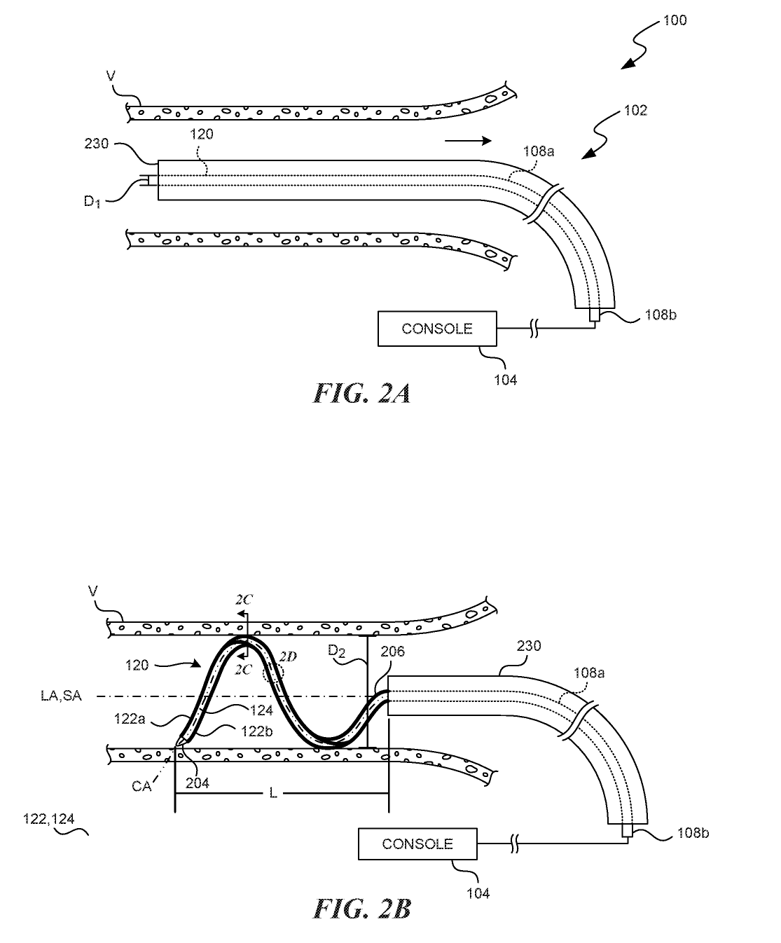

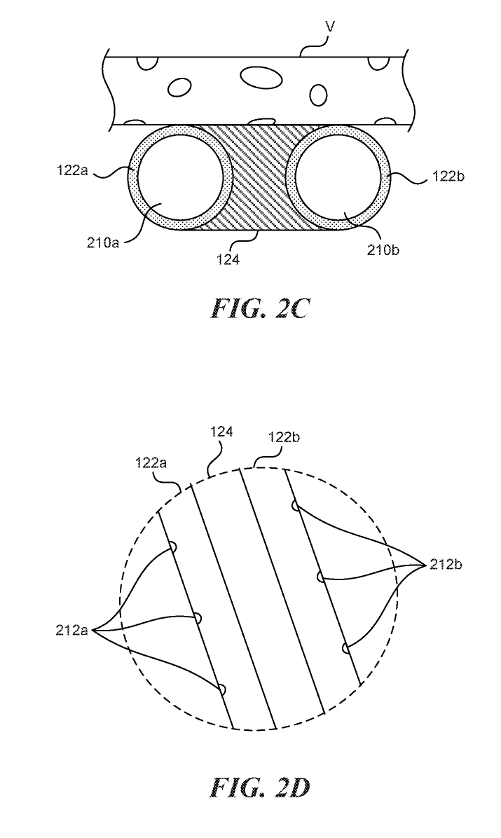

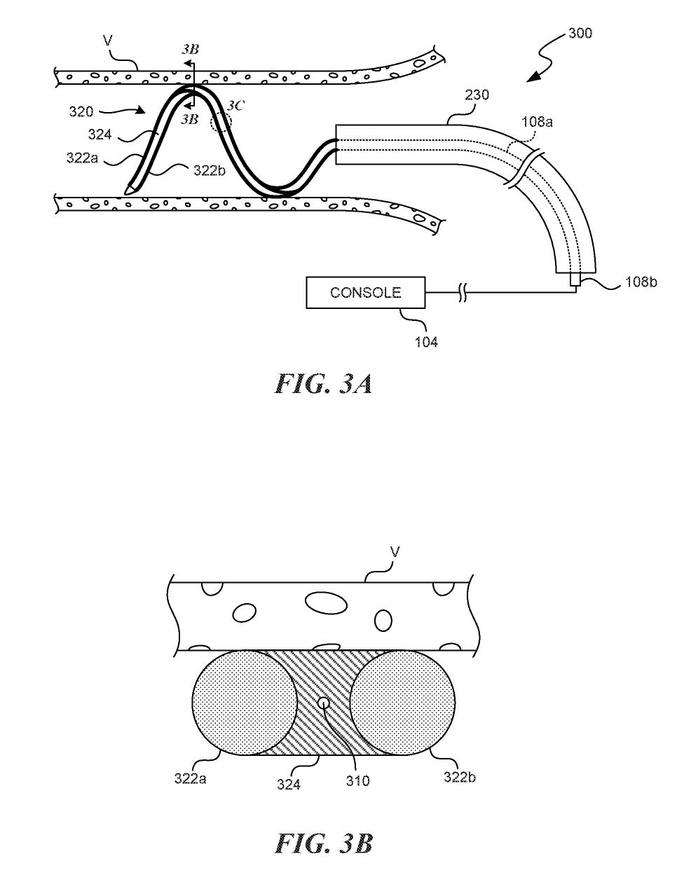

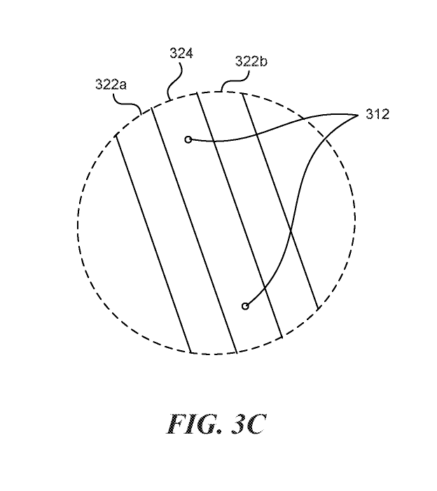

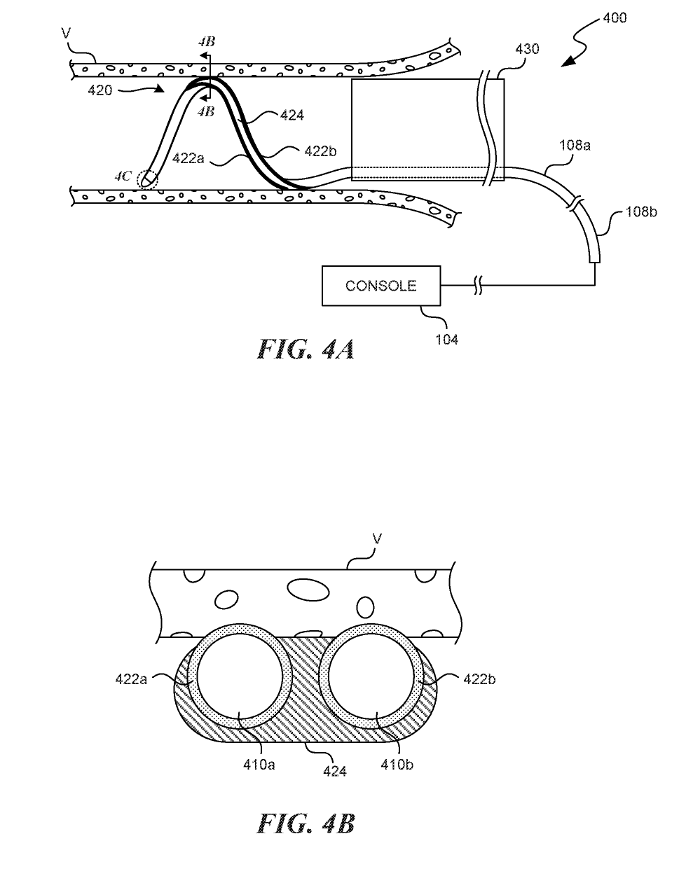

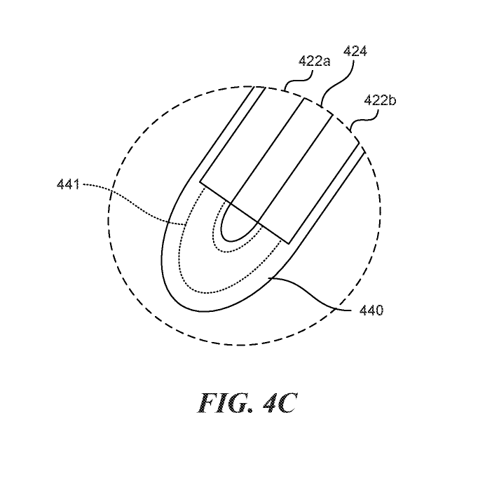

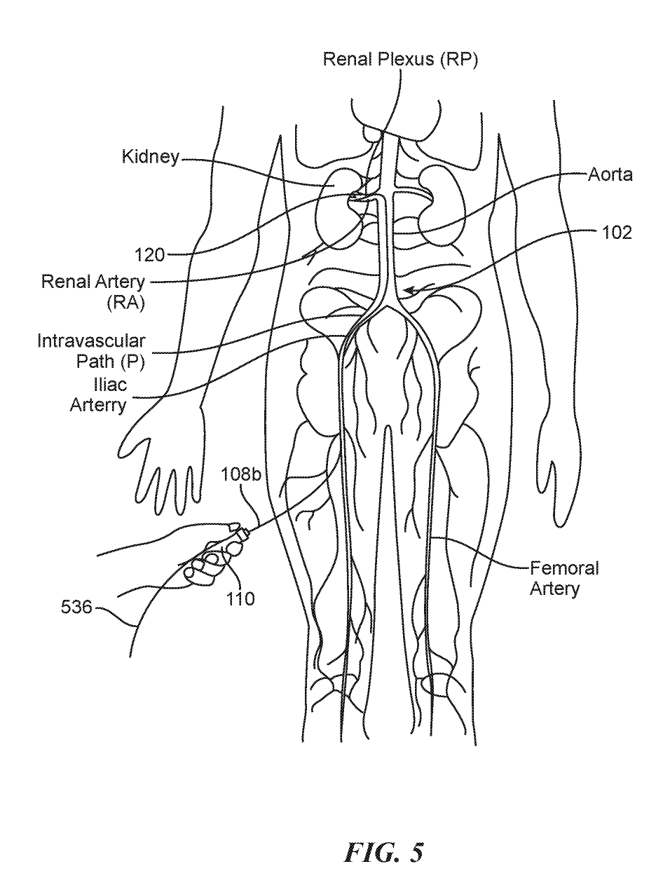

The distal portion 108a of the shaft 108 is configured to be moved within a lumen of a human patient and locate the neuromodulation assembly 120 at a target site within or otherwise proximate to the lumen. For example, shaft 108 can be configured to position the neuromodulation assembly 120 within a blood vessel, a duct, an airway, or another naturally occurring lumen within the human body. In certain embodiments, intravascular delivery of the neuromodulation assembly 120 includes percutaneously inserting a guide wire (see element 536 in FIG. 5) into a body lumen of a patient and moving the shaft 108 and/or the neuromodulation assembly 120 along the guide wire until the neuromodulation assembly 120 reaches a target site (e.g., a renal artery). For example, the distal end of the neuromodulation assembly 120 or other part of the distal portion 108a of the shaft 108 may include a passageway for engaging (e.g., receiving) the guide wire for delivery of the neuromodulation assembly 120 using over-the-wire (OTW) or rapid exchange (RX) techniques. In other embodiments, the neuromodulation catheter 102 can be a steerable or non-steerable device configured for use without a guide wire. In still other embodiments, the neuromodulation catheter 102 can be configured for delivery via a guide catheter or sheath (see element 230 in FIGS. 2B, 3B and element 430 in FIG. 4A).

Once at the target site, the neuromodulation assembly 120 can be configured to apply stimuli, detect resultant hemodynamic responses, and provide or facilitate neuromodulation therapy at the target site (e.g., using the metallic elements 122 and/or other energy delivery elements). For example, the neuromodulation assembly 120 can detect vessel impedance via the metallic elements 122, detect blood flow via a flow sensing element (e.g., a Doppler velocity sensing element (not shown)), detect local blood pressure within the vessel via a pressure transducer or other pressure sensing element (not shown), and/or detect other hemodynamic parameters. The detected hemodynamic responses can be transmitted to the console 104 and/or another device external to the patient. The console 104 can be configured to receive and store the recorded hemodynamic responses for further use by a clinician or operator. For example, a clinician can use the hemodynamic responses received by the console 104 to determine whether an application of neuromodulation energy was effective in modulating nerves to a desired degree.

The console 104 can be configured to control, monitor, supply, and/or otherwise support operation of the neuromodulation catheter 102. The console 104 can further be configured to generate a selected form and/or magnitude of energy for delivery to tissue at the target site via the neuromodulation assembly 120, and therefore the console 104 may have different configurations depending on the treatment modality of the neuromodulation catheter 102. For example, when the neuromodulation catheter 102 is configured for electrode-based, heat-element-based, or transducer-based treatment, the console 104 can include an energy generator (not shown) configured to generate RF energy (e.g., monopolar and/or bipolar RF energy), pulsed electrical energy, microwave energy, optical energy, ultrasound energy (e.g., intravascularly delivered ultrasound, and/or HIFU), direct heat energy, radiation (e.g., infrared, visible, and/or gamma radiation), and/or another suitable type of energy. When the neuromodulation catheter 102 is configured for cryotherapeutic treatment, the console 104 can include a refrigerant reservoir (not shown), and can be configured to supply the neuromodulation catheter 102 with refrigerant. Similarly, when the neuromodulation catheter 102 is configured for chemical-based treatment (e.g., drug infusion), the console 104 can include a chemical reservoir (not shown) and can be configured to supply the neuromodulation catheter 102 with one or more chemicals. In some embodiments, the console 104 can include one or more fluid reservoirs (not shown) for coolant and/or irrigant (e.g., saline) to be delivered to the metallic elements 122 and/or the dielectric material 124 as described in more detail below.

In selected embodiments, the system 100 may be configured to deliver a monopolar electric field via one or more of the metallic elements 122. In such embodiments, a neutral or dispersive electrode 130 may be electrically connected to the console 104 and attached to the exterior of the patient. In embodiments including multiple metallic elements 122, the metallic elements 122 may deliver power independently (i.e., may be used in a monopolar fashion), either simultaneously, selectively, or sequentially, and/or may deliver power between any desired combination of the metallic elements 122 (i.e., may be used in a bipolar fashion). In addition, an operator optionally may be permitted to choose which metallic elements 122 are used for power delivery in order to form customized lesion(s) within the renal artery, as desired. One or more sensing elements (not shown), such as one or more temperature (e.g., thermocouple, thermistor, etc.), pressure, optical, flow, chemical, and/or other sensing elements, may be located proximate to, within, or integral with the metallic elements 122. The sensing element(s) and the metallic elements 122 can be connected to one or more supply wires (not shown) that transmit signals from the sensing element(s) and/or convey energy to the metallic elements 122.

In various embodiments, the system 100 can further include a controller 114 communicatively coupled to the neuromodulation catheter 102. The controller 114 can be configured to initiate, terminate, and/or adjust operation of one or more components (e.g., the metallic elements 122) of the neuromodulation catheter 102 directly and/or via the console 104. In other embodiments, the controller 114 can be omitted or have other suitable locations (e.g., within the handle 110, along the cable 106, etc.). The controller 114 can be configured to execute an automated control algorithm and/or to receive control instructions from an operator. Further, the console 104 can be configured to provide feedback to an operator before, during, and/or after a treatment procedure via an evaluation/feedback algorithm 116.

### Selected Embodiments of Therapeutic Assemblies and Related Devices

FIG. 2A is a partially schematic side view of a portion of the system 100 in a delivery state (e.g., the neuromodulation assembly 120 in a low-profile or collapsed configuration) and FIG. 2B is a partially schematic side view of a portion of the system 100 of FIG. 2A in a deployed state (e.g., the neuromodulation assembly 120 in an expanded configuration). As noted above, the neuromodulation assembly 120 disposed at the distal portion 108a of the elongated shaft 108 can be transformed or actuated between the delivery state as shown in FIG. 2A and the deployed state (e.g., a radially expanded, generally spiral/helical configuration, as shown in FIG. 2B). FIG. 2C is a cross-sectional view of the neuromodulation assembly 120 taken along line 2C-2C in FIG. 2B, and FIG. 2D is an enlarged detail view of a portion of the neuromodulation system shown in FIG. 2B.

Referring to FIGS. 2A-2D together, the neuromodulation assembly 120 is positioned at a target site within a blood vessel V (e.g., a renal artery) of a human patient. As best shown in FIG. 1, the catheter 102 includes elongated shaft 108, which has a distal portion 108a configured to be positioned at the target site within the blood vessel V and a proximal portion 108b that extends outside of the patient to a handle (not shown) or other feature that allows an operator to manipulate the distal portion 108a. One or more portions of the elongated shaft 108 can comprise a solid wire and/or a wire coil. For example, in some embodiments, the proximal portion 108b of the elongated shaft 108 comprises a solid wire and the distal portion 108a comprises two or more shape-memory metallic elements as described in more detail below. Additionally, the elongated shaft 108 can have a uniform stiffness along its length, or can have a stiffness that varies along its length.

FIG. 2A illustrates the neuromodulation assembly 120 constrained in the delivery state (e.g., a low profile or collapsed configuration) within the lumen of a tubular sheath or delivery element 230. The delivery element 230 may be sized to fit slidably through a guide catheter (not shown) and have a lumen sized and shaped to radially restrain the neuromodulation assembly 120 in the low-profile state for delivery to the target treatment site within the vessel V. In the collapsed, low-profile configuration, the neuromodulation assembly 120 is configured to move through the delivery element 230 to the treatment site. In some embodiments, the delivery element 230 may be sized to slidably fit through an 8 Fr or smaller guide catheter to accommodate small arteries (e.g. radial artery) during delivery of the neuromodulation assembly 120 to the treatment site. In other embodiments, however, the delivery element 230 may have a different size.

FIG. 2B illustrates the neuromodulation assembly 120 after the delivery element 230 has been proximally retracted from the distal portion 108a. As illustrated, when not constrained by the delivery element 230, the neuromodulation assembly 120 assumes an expanded, generally helical shape. The dimensions (e.g., outer diameter and length) of the spiral/helical portion of the neuromodulation assembly 120 can be selected to accommodate the vessels or other body lumens in which the neuromodulation assembly 120 is designed to be delivered. For example, the axial length of the spiral/helical portion of the neuromodulation assembly 120 may be selected to be no longer than a patient's renal artery (e.g., typically less than 7 cm), and have a diameter that accommodates the inner diameter of a typical renal artery (e.g., about 2-10 mm). For other clinical applications, such as treatment in a pulmonary vein, the spiral/helical portion of the neuromodulation assembly 120 can have other dimensions depending on the body lumen within which it is configured to be deployed. In further embodiments, the neuromodulation assembly 120 can have other suitable shapes (e.g., semi-circular, curved, straight, etc.). The neuromodulation assembly 120 may also be designed to apply a desired outward radial force to a vessel when expanded to the spiral/helical deployed state (shown in FIG. 2B) to be in contact with the vessel wall. This effect is achieved by the assembly 120 having a pre-formed helical shape slightly larger than the vessel lumen at the top of the range of intended target vessel sizes.

As best seen in FIGS. 2C and 2D, at least two metallic elements 122a and 122b (collectively metallic elements 122) extend parallel to each other along at least a portion of the length of the neuromodulation assembly 120. The dielectric material 124 is an elongated element that also extends along at least a portion of the length of the neuromodulation assembly 120 between the metallic elements 122. The dielectric material 124 attaches the metallic elements 122 to each other while maintaining physical separation and electrical isolation. In an embodiment, the metallic elements 122 are parallel as a result of their physical separation distance being held constant by the dielectric material 124. In one embodiment, the metallic elements 122 extend along the full length of the neuromodulation assembly 120, and the dielectric material 124 binds the metallic elements 122 together along the full length of the metallic elements 122. The metallic elements 122 can be made of shape-memory material that has been pre-formed to impart a spiral/helical shape to the neuromodulation assembly 120 when it is not radially constrained. As used herein, shape-memory material may be nitinol, a nickel titanium alloy having stress-induced martensite (SIM) properties that are also referred to as superelasticity or pseudo-elasticity. Although the heat-triggered, thermal shape memory properties of nitinol may be used in the invention, it is the elastic properties that permit nitinol to return to a pre-formed shape without being heated that are considered most useful in making metallic elements 122. Other electrically conductive elastic materials such as spring-tempered stainless steel may also be used as a material for metallic elements 122.

In the embodiment shown in FIGS. 2A and 2B, the neuromodulation assembly 120 is configured such that, in the expanded state (FIG. 2B), the metallic elements 122 form parallel helices around a longitudinal axis LA of the neuromodulation assembly 120. The distance separating the metallic elements 122 may remain unchanged between the low-profile, constrained configuration (FIG. 2A) and the expanded configuration (FIG. 2B). In some embodiments, the metallic elements 122 and the separating dielectric material 124 extend along the entire length of the catheter 102, while in other embodiments the metallic elements 122 and/or the separating dielectric 124 are limited to the full length or only a portion of the full length of the neuromodulation assembly 120. The dielectric material 124 can partially encapsulate the metallic elements 122 leaving exposed regions in which the metallic elements are not covered by the dielectric material 124. These exposed regions can deliver energy from the metallic elements 122 to a target site, while the encapsulated regions are insulated by the dielectric material 124 which can prevent other areas from directly receiving energy from the metallic elements 122. In an embodiment (FIG. 4B), the exposed regions of the metallic elements 122 face outwardly when in the expanded state such that the exposed regions of the metallic elements 122 are in contact with the inner wall of the vessel V and the encapsulated regions face radially inwardly towards the center of the vessel V. As described in more detail below, in some embodiments the metallic elements are tubes with lumens configured to carry fluid therein, or in other embodiments the metallic elements are solid wires.

In one embodiment, the metallic elements 122 can each be a tubular structure comprising a nitinol multifilar stranded wire with a lumen therethrough and sold under the trademark HELICAL HOLLOW STRAND (HHS), and commercially available from Fort Wayne Metals of Fort Wayne, Ind. The metallic elements 122 may be formed from a variety of different types of materials, may be arranged in a single or dual-layer configuration, and may be manufactured with a selected tension, compression, torque and pitch direction. The HHS material, for example, may be cut using a laser, electrical discharge machining (EDM), electrochemical grinding (ECG), or other suitable means to achieve a desired finished component length and geometry.

Forming the metallic elements 122 of nitinol multifilar stranded wire(s) or other similar materials is expected to provide a desired level of support and rigidity to the neuromodulation assembly 120 without additional reinforcement wire(s) or other reinforcement features. This feature is expected to reduce the number of manufacturing processes required to form the neuromodulation assembly 120 and reduce the number of materials required for the device. In one embodiment, the pre-formed spiral shape is formed from a shape memory material (e.g., nickel-titanium (nitinol)) wire or tube that is shaped around a mandrel (not shown). In one specific example, nitinol shape memory wire can typically be heated for approximately 510° C. for approximately 5 minutes followed by a water quench. After the wires are formed into the spiral shape, they can be held in place relative to one another (e.g., extending in parallel in a generally helical shape) while a dielectric material is provided in the space between the wires.

In one embodiment, the dielectric material 124 separating the metallic elements 122 electrically isolates the metallic elements 122 from each other. The dielectric material 124 may be composed of a polymer material such as polyamide, polyimide, polyether block amide copolymer sold under the trademark PEBAX, polyethylene terephthalate (PET), polypropylene, an aliphatic, polycarbonate-based thermoplastic polyurethane sold under the trademark CARBOTHANE, or a polyether ether ketone (PEEK) polymer or other suitable materials. The material properties and dimensions of the dielectric material 124 are selected to provide the necessary flexibility for the neuromodulation assembly 120 to transform between a radially constrained, substantially straight shape and a relaxed shape that tends to conform to the spiral/helical shape of the pre-formed metallic elements 122. In other words, the dielectric material 124 is more flexible than the metallic elements 122 such that the shape of the combined components is defined in large part by the shape of the metallic elements 122.

In other embodiments, the metallic elements 122 and/or other components of the neuromodulation assembly 120 may be composed of different materials and/or have a different arrangement. For example, the metallic elements 122 may be formed from other suitable shape memory materials (e.g., wire or tubing besides HHS or nitinol, shape memory polymers, electro-active polymers) that are pre-formed or pre-shaped into the desired deployed state. Alternatively, the metallic elements 122 may be formed from multiple materials such as a composite of one or more polymers and metals.

As best seen in FIG. 2B, after delivery to the target treatment site (e.g. renal artery RA), the neuromodulation assembly 120 may be deployed to an expanded, spiral-shaped configuration with the metallic elements 122 in contact with the vessel wall. In one embodiment, for example, the neuromodulation assembly 120 is deployed by retracting the delivery element 230, releasing the metallic elements 122 to expand radially toward their pre-formed spiral shape and thereby define an imaginary cylinder around a central longitudinal axis LA of the vessel V. As shown in FIG. 2B, the pre-formed spiral shape facilitates radial expansion in a direction toward the inner wall of the vessel V such that the metallic elements 122 contact and press outwardly against the inner wall. The metallic elements 122 are configured to assume an expanded configuration when in an unbiased (e.g., unconstrained) condition in such embodiments.

As shown in FIG. 2B where vessel V has a lumen diameter D₂ that is at least slightly smaller than the pre-formed diameter of the spiral/helix configuration of the neuromodulation assembly 120, the spiral/helix configuration can be characterized, at least in part, by the radially-expanded outer dimension D₂, length L₁, pitch (longitudinal distance of one complete helix turn measured parallel to a central spiral axis SA), and number of revolutions (number of times the helix completes a 360° revolution about the central spiral axis SA). When expanded in free space, e.g., not restricted by a vessel wall, a delivery element or other structure, the spiral/helical configuration of the neuromodulation assembly 120 may be characterized by its free diameter, free axial length, free pitch and number of revolutions. As the neuromodulation assembly 120 expands from its delivery state, its low-profile outer dimension D₁ (FIG. 2A) increases to a vessel-defined radially expanded outer dimension D₂ (FIG. 2B) and its length decreases. That is, when the neuromodulation assembly 120 deploys into a spiral/helical shape, a distal end 204 moves axially towards the proximal end 206 (or vice versa). Accordingly, the deployed length is less than the unexpanded or delivery length.

Upon deployment, the pre-formed spiral shape provides a curvilinear axis CA about the central spiral axis SA. Referring to FIG. 2C, the neuromodulation assembly 120 is configured to press a first face of each of the metallic elements 122a and 122b against an interior wall of the blood vessel V for delivering therapeutically effective energy to target tissue (e.g., one or more nerves) of the patient. For example, when the neuromodulation assembly 120 is deployed in the vessel V of the patient, the central spiral axis SA is generally aligned with the central longitudinal axis LA of the vessel V such that the pre-formed spiral shape (e.g., the curvilinear axis CA) positions the metallic elements 122 in stable apposition with the interior wall of the vessel V.

In one embodiment, the individual metallic elements 122 can be electrodes configured to deliver energy (e.g., electrical energy, RF energy, pulsed electrical energy, non-pulsed electrical energy, thermal energy, etc.) across the wall of the vessel V. In a specific embodiment, each metallic element 122a, 122b in conjunction with a neutral electrode 130 can deliver a monopolar thermal RF field to targeted renal nerves adjacent the wall of the vessel V. Alternatively, a bipolar thermal RF field generated between metallic elements 122a and 122b can be delivered to targeted renal nerves adjacent the wall of the vessel V. The metallic elements 122 are electrically connected to an external energy source such as the console 104 by conductor or bifilar wires (not shown) extending through catheter 102. In some embodiments the metallic elements 122 themselves extend along the entire length of the catheter 102, while in other embodiments the metallic elements 122 are limited to the distal portion 108a. In some embodiments, the metallic elements 122 may be welded or otherwise electrically coupled to their energy supply wires, and the wires can extend the entire length of the catheter 102 such that a proximal end thereof is coupled to the console 104.

In operation and referring to FIGS. 1-2D together, after the neuromodulation assembly 120 is self-expanded or otherwise deployed to a vessel-restricted spiral/helical configuration with the metallic elements 122 in apposition with the interior wall of the renal artery RA, therapeutically-effective energy can be delivered via the metallic elements 122 across the wall of the renal artery RA to targeted renal nerves (not shown) at one or more treatment locations. In one embodiment, the metallic elements 122a and 122b are electrically biased at opposite polarities to provide a localized bipolar electrical field along the neuromodulation assembly 120. In another embodiment, the metallic elements 122a and 122b are electrically biased at common polarity and a return electrode is located elsewhere to provide a monopolar field.

The metallic elements 122a, 122b can be electrically conductive tubes that each include a hollow lumen 210a, 210b, respectively, and apertures 212a, 212b, respectively, in the sidewall of the tubes. The lumens 210a, 210b are in fluid communication with apertures 212a, 212b such that an irrigating fluid (e.g., saline) can be emitted from the metallic elements 122a, 122b via the apertures 212a, 212b for irrigation of the treatment site during neuromodulation.

After forming sufficient lesions or treatment zones to achieve neuromodulation, and in accordance with one method, the system 100 may be transformed back to the low-profile delivery state by distally advancing the delivery element 230 relative to the neuromodulation assembly 120. Once the delivery element 230 is in position at the treatment site and the neuromodulation assembly 120 is re-constrained in the low-profile delivery state, the system 100 can be pulled back out of the vessel V.

FIG. 3A is a partially schematic side view of another embodiment of a neuromodulation system 300 in an expanded state and positioned within a blood vessel of a human patient. FIG. 3B is a cross-sectional view of the system 300 taken along line 3B-3B in FIG. 3A, and FIG. 3C is an enlarged detail view of a portion of the system 300 shown in FIG. 3A. Referring to FIGS. 3A-3C together, a neuromodulation assembly 320 can include several features generally similar to the neuromodulation assembly 120 described above with respect to FIGS. 1-2D. For example, the neuromodulation assembly 320 includes elongated metallic elements 322a, 322b separated by a dielectric material 324. The metallic elements 322a, 322b and the dielectric material 324 can be as described above with respect to metallic elements 122a, 122b, and dielectric material 124, except that the metallic elements 322a, 322b are solid wires rather than tubes with hollow lumens as in the case of metallic elements 122a, 122b. As best shown in FIG. 3B, the dielectric material 324 of this embodiment includes a lumen 310 extending along the length of the neuromodulation assembly 320. The lumen 310 can be in fluid communication with a fluid source containing, for example, saline or other irrigating fluid. The neuromodulation assembly 320 further includes a plurality of apertures 312 in the dielectric material 324 along its length that are in fluid communication with the lumen 310. In operation, irrigating fluid is delivered through the lumen 310 and is emitted via the apertures 312 into surrounding areas to assist in irrigating the treatment site during neuromodulation.

FIG. 4A is a partially schematic side view of yet another embodiment of a neuromodulation system 400 in an expanded state and positioned within a blood vessel of a human patient. FIG. 4B is a cross-sectional view of the system 400 taken along line 4B-4B in FIG. 4A, and FIG. 4C is an enlarged detail view of a distal portion of the system 400 shown in FIG. 4A. Referring to FIGS. 4A-4C together, a neuromodulation assembly 420 can include several features generally similar to the neuromodulation assembly 120 described above with respect to FIGS. 1-2D. For example, the neuromodulation assembly 420 includes elongated metallic elements 422a, 422b separated by a dielectric material 424. The metallic elements 422a, 422b can be as described above with respect to metallic elements 122a, 122b. The dielectric material 424 can have some similarities to the dielectric material 124 described above, except that the dielectric material 424 covers a greater portion of the metallic elements 422a, 422b. More specifically, in one embodiment the dielectric material 424 covers a radially-inward facing portion of the metallic elements 422a, 422b when the neuromodulation assembly 420 is unconstrained and assumes a spiral shape. By covering the radially-inwardly facing portions of the metallic elements 422a, 422b, the energy is not delivered directly to the blood flowing through the vessel, but rather the energy is directed primarily to the tissue of the vessel contacting the metallic elements 422a, 422b. As a result, less power may be used to achieve sufficient results compared other embodiments.

In the embodiments shown in FIGS. 4A-4C, the metallic elements 422a, 422b are conductive tubes having lumens 410a, 410b, respectively, that can be coupled to a fluid coolant source at a proximal end (not shown) and a connector 440 at a distal end. The connector 440 is in fluid communication with both the lumens 410a, 410b and includes an inner flow path 441 connecting the two so that fluid can be circulated through the neuromodulation assembly 420. The connector 440 can be made of the same material as dielectric material 424, or any other suitable material. This configuration provides for circulating coolant to carry heat away from the metallic elements 422a, 422b during neuromodulation at the treatment site.

In another embodiment, the metallic elements 422a, 422b of the neuromodulation assembly 420 can be solid wires similar to the solid wires 322a, 322b described above. In this embodiment, the dielectric material 424 can have a lumen and apertures for delivering an irrigation fluid as described above with reference to the dielectric material 324, lumen 310, and apertures 312 described above with respect to FIG. 3.

As shown in FIG. 4A, a delivery element 430 used to deliver the neuromodulation assembly 420 to the treatment site can include an off-set inner lumen. This configuration delivers the neuromodulation assembly 420 closer to one side of the vessel V than another, which promotes apposition of the metallic elements 422a, 422b with the inner wall of the vessel V when in the expanded configuration as the metallic elements 422a, 422b immediately contact the inner wall of the vessel V upon exiting the delivery element 430.

### Renal Neuromodulation

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, chemically-induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

Renal neuromodulation can include a cryotherapeutic treatment modality alone or in combination with another treatment modality. Cryotherapeutic treatment can include cooling tissue at a target site in a manner that modulates neural function. For example, sufficiently cooling at least a portion of a sympathetic renal nerve can slow or potentially block conduction of neural signals to produce a prolonged or permanent reduction in renal sympathetic activity. This effect can occur as a result of cryotherapeutic tissue damage, which can include, for example, direct cell injury (e.g., necrosis), vascular or luminal injury (e.g., starving cells from nutrients by damaging supplying blood vessels), and/or sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Neuromodulation using a cryotherapeutic treatment in accordance with embodiments of the present technology can include cooling a structure proximate an inner surface of a body lumen wall such that tissue is effectively cooled to a depth where sympathetic renal nerves reside. For example, in some embodiments, a cooling assembly of a cryotherapeutic device can be cooled to the extent that it causes therapeutically-effective, cryogenic renal neuromodulation. In other embodiments, a cryotherapeutic treatment modality can include cooling that is not configured to cause neuromodulation. For example, the cooling can be at or above cryogenic temperatures and can be used to control neuromodulation via another treatment modality (e.g., to protect tissue from neuromodulating energy).

Renal neuromodulation can include an electrode-based or transducer-based treatment modality alone or in combination with another treatment modality. Electrode-based or transducer-based treatment can include delivering electricity and/or another form of energy to tissue at a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at a treatment location. For example, neuromodulation in accordance with embodiments of the present technology can include delivering RF energy, pulsed electrical energy, microwave energy, optical energy, focused ultrasound energy (e.g., high-intensity focused ultrasound energy), or another suitable type of energy alone or in combination. An electrode or transducer used to deliver this energy can be used alone or with other electrodes or transducers in a multi-electrode or multi-transducer array. Furthermore, the energy can be applied from within the body (e.g., within the vasculature or other body lumens in a catheter-based approach) and/or from outside the body (e.g., via an applicator positioned outside the body). Furthermore, energy can be used to reduce damage to non-targeted tissue when targeted tissue adjacent to the non-targeted tissue is subjected to neuromodulating cooling.

Neuromodulation using focused ultrasound energy (e.g., high-intensity focused ultrasound energy) can be beneficial relative to neuromodulation using other treatment modalities. Focused ultrasound is an example of a transducer-based treatment modality that can be delivered from outside the body. Focused ultrasound treatment can be performed in close association with imaging (e.g., magnetic resonance, computed tomography, fluoroscopy, ultrasound (e.g., intravascular or intraluminal), optical coherence tomography, or another suitable imaging modality). For example, imaging can be used to identify an anatomical position of a treatment location (e.g., as a set of coordinates relative to a reference point). The coordinates can then entered into a focused ultrasound device configured to change the power, angle, phase, or other suitable parameters to generate an ultrasound focal zone at the location corresponding to the coordinates. The focal zone can be small enough to localize therapeutically-effective heating at the treatment location while partially or fully avoiding potentially harmful disruption of nearby structures. To generate the focal zone, the ultrasound device can be configured to pass ultrasound energy through a lens, and/or the ultrasound energy can be generated by a curved transducer or by multiple transducers in a phased array (curved or straight).

Without being bound by theory, the heating effects of electrode-based or transducer-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. It is expected that heating tissue to a temperature between about body temperature and about 45° C. can induce non-ablative alteration, for example, via moderate heating of target neural fibers or of vascular or luminal structures that perfuse the target neural fibers. In such cases where vascular structures are affected, the target neural fibers can be denied perfusion resulting in necrosis of the neural tissue. Alternatively, heating tissue to a target temperature higher than about 45° C. (e.g., higher than about 60° C.) can induce ablation, for example, via substantial heating of target neural fibers or of vascular or luminal structures that perfuse the target fibers. In some patients, it can be desirable to heat tissue to temperatures that are sufficient to ablate the target neural fibers or the vascular or luminal structures, but that are less than about 90° C. (e.g., less than about 85° C., less than about 80° C., or less than about 75° C.).

Renal neuromodulation can include a chemical-based treatment modality alone or in combination with another treatment modality. Neuromodulation using chemical-based treatment can include delivering one or more chemicals (e.g., drugs or other agents) to tissue at a treatment location in a manner that modulates neural function. The chemical, for example, can be selected to affect the treatment location generally or to selectively affect some structures at the treatment location over other structures. The chemical, for example, can be guanethidine, ethanol, phenol, a neurotoxin, or another suitable agent selected to alter, damage, or disrupt nerves. A variety of suitable techniques can be used to deliver chemicals to tissue at a treatment location. For example, chemicals can be delivered via one or more needles originating outside the body or within the vasculature or other body lumens. In an intravascular example, a catheter can be used to intravascularly position a therapeutic element including a plurality of needles (e.g., micro-needles) that can be retracted or otherwise blocked prior to deployment. In other embodiments, a chemical can be introduced into tissue at a treatment location via simple diffusion through a body lumen wall, electrophoresis, or another suitable mechanism. Similar techniques can be used to introduce chemicals that are not configured to cause neuromodulation, but rather to facilitate neuromodulation via another treatment modality.

FIG. 5 (with additional reference to FIG. 1) illustrates modulating renal nerves in accordance with an embodiment of the system 100. The neuromodulation catheter 102 provides access to the renal plexus RP through an intravascular path P, such as a percutaneous access site in the femoral (illustrated), brachial, radial, or axillary artery to a targeted treatment site within a respective renal artery RA. By manipulating the proximal portion 108b of the shaft 108 from outside the intravascular path P, a clinician may advance the shaft 108 through the sometimes tortuous intravascular path P and remotely manipulate the distal portion 108a (FIG. 1) of the shaft 108. In the embodiment illustrated in FIG. 5, the neuromodulation assembly 120 is delivered intravascularly to the treatment site using a guide wire 536 in an OTW technique. As noted previously, the distal end of the neuromodulation assembly 120 may define a passageway for receiving the guide wire 536 for delivery of the neuromodulation catheter 102 using either OTW or RX techniques. At the treatment site, the guide wire 536 can be at least partially withdrawn or removed, and the neuromodulation assembly 120 can transform or otherwise be moved to a deployed arrangement for recording neural activity and/or delivering energy at the treatment site. In other embodiments, the neuromodulation assembly 120 may be delivered to the treatment site within a guide sheath (not shown) with or without using the guide wire 536. When the neuromodulation assembly 120 is at the target site, the guide sheath may be at least partially withdrawn or retracted and the neuromodulation assembly 120 can be transformed into the deployed arrangement. In still other embodiments, the shaft 108 may be steerable itself such that the neuromodulation assembly 120 may be delivered to the treatment site without the aid of the guide wire 536 and/or guide sheath.

Image guidance, e.g., computed tomography (CT), fluoroscopy, intravascular ultrasound (IVUS), optical coherence tomography (OCT), intracardiac echocardiography (ICE), or another suitable guidance modality, or combinations thereof, may be used to aid the clinician's positioning and manipulation of the neuromodulation assembly 120. For example, a fluoroscopy system (e.g., including a flat-panel detector, x-ray, or c-arm) can be rotated to accurately visualize and identify the target treatment site. In other embodiments, the treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the neuromodulation assembly 120. Further, in some embodiments, image guidance components (e.g., IVUS, OCT) may be integrated with the neuromodulation catheter 102 and/or run in parallel with the neuromodulation catheter 102 to provide image guidance during positioning of the neuromodulation assembly 120. For example, image guidance components (e.g., IVUS or OCT) can be coupled to the neuromodulation assembly 120 to provide three-dimensional images of the vasculature proximate the target site to facilitate positioning or deploying the multi-electrode assembly within the target renal blood vessel.

Energy from the metallic elements 122 (FIG. 1) and/or other energy delivery elements may then be applied to target tissue to induce one or more desired neuromodulating effects on localized regions of the renal artery RA and adjacent regions of the renal plexus RP, which lay intimately within, adjacent to, or in close proximity to the adventitia of the renal artery RA. The purposeful application of the energy may achieve neuromodulation along all or at least a portion of the renal plexus RP. The neuromodulating effects are generally a function of, at least in part, power, time, contact between the energy delivery elements and the vessel wall, and blood flow through the vessel. The neuromodulating effects may include denervation, thermal ablation, and/or non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 45° C. for non-ablative thermal alteration, or the target temperature may be about 45° C. or higher for the ablative thermal alteration. Desired non-thermal neuromodulation effects may include altering the electrical signals transmitted in a nerve.

### Related Anatomy and Physiology

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

A. The Sympathetic Chain

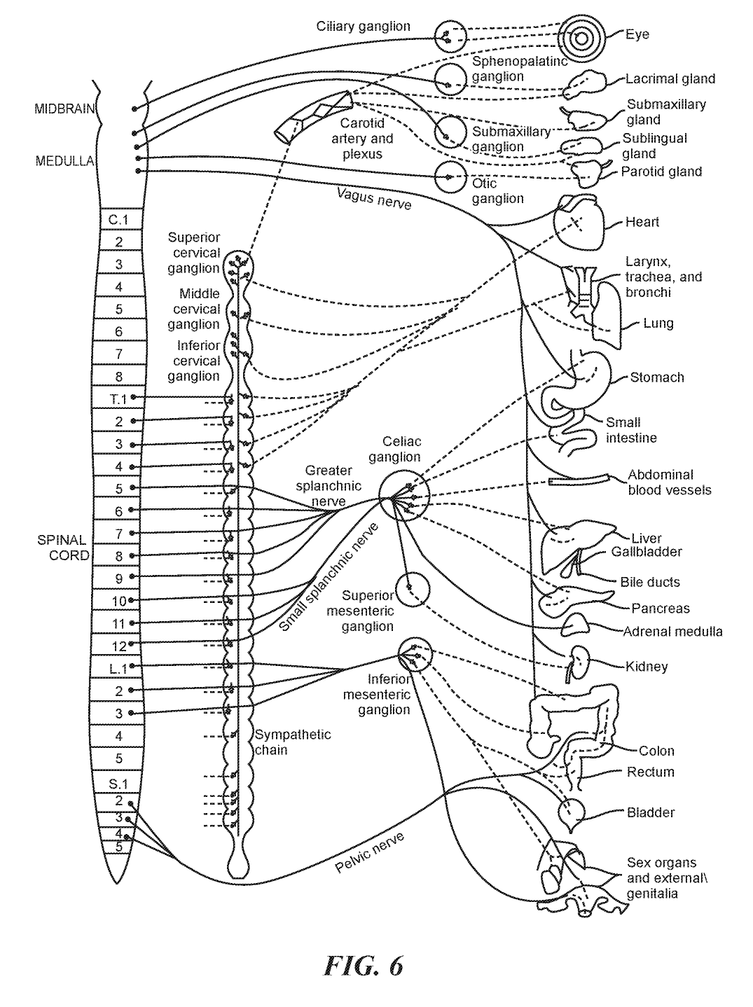

As shown in FIG. 6, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

1. Innervation of the Kidneys

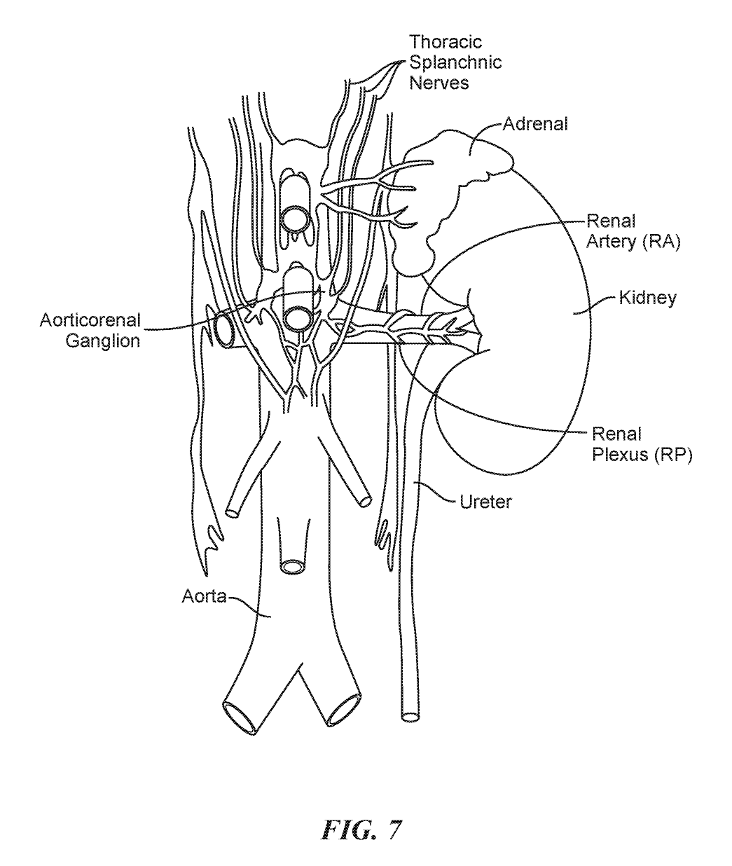

As FIG. 7 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

2. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

(i) Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

(ii) Renal Sensory Afferent Nerve Activity

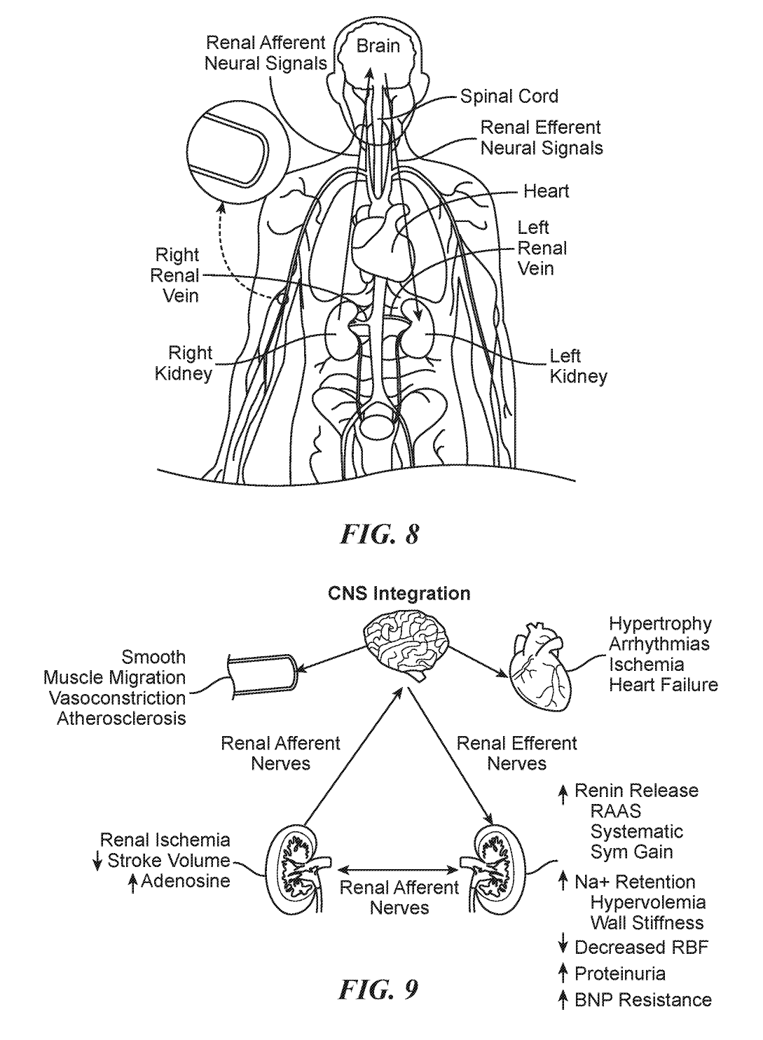

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 8 and 9, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 7. For example, as previously discussed, a reduction in central sympathetic drive may reduce the insulin resistance that afflicts people with metabolic syndrome and Type II diabetics. Additionally, patients with osteoporosis are also sympathetically activated and might also benefit from the down regulation of sympathetic drive that accompanies renal denervation.

C. Achieving Intravascular Access to the Renal Artery

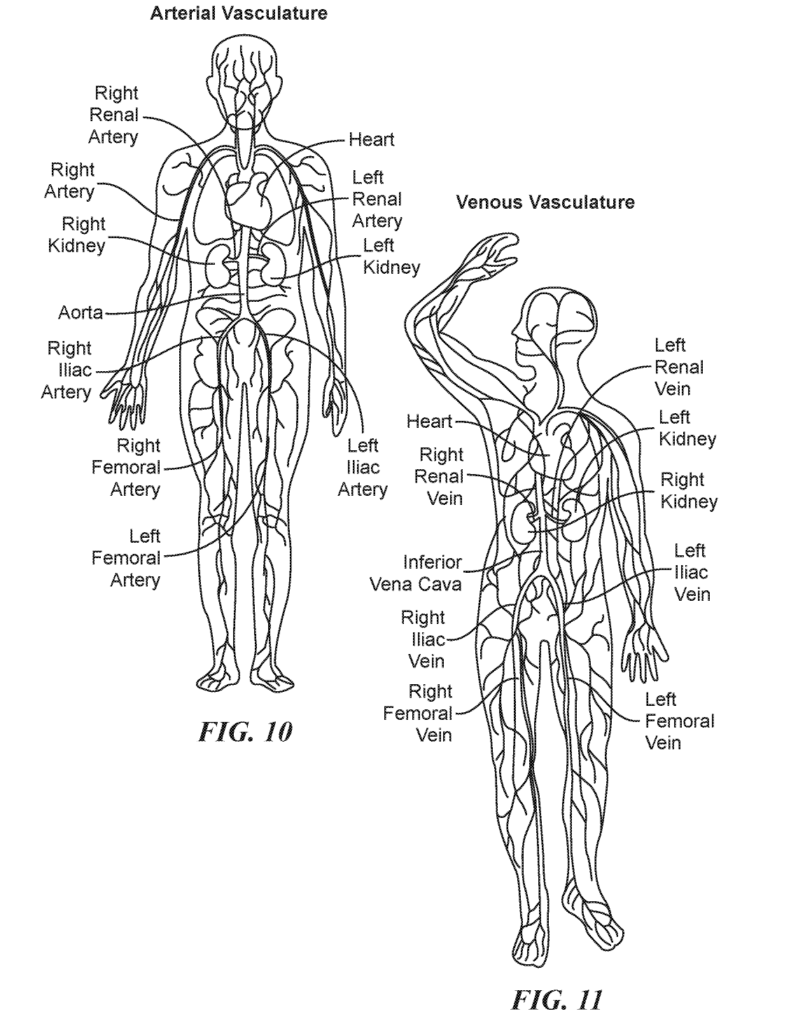

In accordance with the present technology, neuromodulation of a left and/or right renal plexus (RP), which is intimately associated with a left and/or right renal artery, may be achieved through intravascular access. As FIG. 10 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 11 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e. cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, a full-circle lesion likely resulting from a continuous circumferential treatment may be potentially related to renal artery stenosis. Therefore, the formation of more complex lesions along a longitudinal dimension of the renal artery and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery should also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time should be avoided because to prevent injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility; and (f) the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta induced by respiration and/or blood flow pulsatility. A patient's kidney, which is located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

I/We claim:

1. A neuromodulation catheter, comprising:
a therapeutic assembly disposed at a distal portion of the neuromodulation catheter and adapted to be located at a target location within a target blood vessel of a human patient, the distal portion having a length, and the therapeutic assembly including
at least two shape-memory metallic elements that extend parallel to each other along at least a portion of the length of the distal portion, the metallic elements being configured to transform the therapeutic assembly between a low-profile delivery configuration and a deployed radially-expanded spiral configuration; and
a dielectric material separating the metallic elements along at least the distal portion.

2. The neuromodulation catheter of claim 1, wherein the therapeutic assembly further comprises a sheath configured to extend over at least a portion of the metallic elements, wherein the metallic elements are configured to transform between the low-profile delivery configuration when constrained by the sheath and the deployed expanded spiral configuration when the sheath is retracted.

3. The neuromodulation catheter of claim 1 wherein the metallic elements comprise electrodes.

4. The neuromodulation catheter of claim 1 wherein the metallic elements comprise nitinol elements.

5. The neuromodulation catheter of claim 1 wherein the metallic elements further extend along a portion of the neuromodulation catheter proximal to the distal portion.

6. The neuromodulation catheter of claim 1 wherein the dielectric material partially encapsulates the metallic elements leaving a plurality of exposed regions in which the metallic elements are not covered by the dielectric material.

7. The neuromodulation catheter of claim 1 wherein the metallic elements comprise hollow wires.

8. The neuromodulation catheter of claim 7 wherein the hollow wires are configured to receive irrigating fluid therethrough, the hollow wires including a plurality of apertures along their lengths that permit irrigating fluid to be emitted from the hollow wires.

9. The neuromodulation catheter of claim 7 wherein the hollow wires are in fluid communication with one another via a connector disposed at distal ends of each of the hollow wires, wherein the hollow wires and the connector are configured to circulate a coolant fluid therethrough.

10. The neuromodulation catheter of claim 1 wherein the dielectric material comprises a lumen therein configured to receive irrigating fluid therethrough, the lumen including a plurality of apertures along its length that permit irrigating fluid to be emitted from the lumen.

11. The neuromodulation catheter of claim 1 wherein, when the therapeutic assembly is in the deployed radially-expanded spiral configuration, the metallic elements follow a curvilinear axis.

12. The neuromodulation catheter of claim 1 wherein, when the therapeutic assembly is in the deployed expanded spiral configuration, the metallic elements form parallel helices around a longitudinal axis of the catheter.

13. The neuromodulation catheter of claim 1 wherein the dielectric material covers a first portion of each of the metallic elements and does not cover a second portion of each of the metallic elements, and wherein, when the therapeutic assembly assumes the deployed expanded spiral configuration, the first portions of the metallic elements face radially inwardly and the second portions of the metallic elements face radially outwardly.

14. A neuromodulation catheter having a distal portion with a length adapted to be located at a target location within a target blood vessel of a human patient, the neuromodulation catheter comprising:
at least two elongated conductive elements that both extend longitudinally along a common portion of the length of the distal portion;
a dielectric element extending longitudinally along the common portion of the length of the distal portion and separating the conductive elements; and
a sheath configured to removably constrain the conductive elements, wherein the conductive elements tend to form a helical configuration when released from the sheath.

15. The neuromodulation catheter of claim 14 wherein the conductive elements are parallel to each other in the common portion of the length of the distal portion.

16. The neuromodulation catheter of claim 14 wherein the common portion comprises the entire length of the distal portion.

17. The neuromodulation catheter of claim 14 wherein the common portion comprises less than the entire length of the distal portion.

18. The neuromodulation catheter of claim 14 wherein the conductive elements comprise a shape-memory material.

19. The neuromodulation catheter of claim 14 wherein the conductive elements further extend along a portion of the neuromodulation catheter proximal to the distal portion.

20. The neuromodulation catheter of claim 14 wherein the conductive elements comprise hollow wires configured to receive fluid therethrough.

21. The neuromodulation catheter of claim 20 wherein the hollow wires include a plurality of apertures along their lengths that permit fluid to be emitted from the hollow wires.

22. The neuromodulation catheter of claim 20 wherein the hollow wires are in fluid communication with one another via a connector disposed at distal ends of each of the hollow wires, wherein the hollow wires and the connector are configured to circulate fluid therethrough.

23. The neuromodulation catheter of claim 14 wherein the dielectric element comprises a lumen therein configured to receive irrigating fluid therethrough, the lumen including a plurality of apertures along its length that permit fluid to be emitted from the lumen.

24. The neuromodulation catheter of claim 14 wherein in the helical configuration, the conductive elements follow a curvilinear axis and the helical configuration is sized for apposition with an inner wall of the target blood vessel.

25. The neuromodulation catheter of claim 14 wherein in the helical configuration, the conductive elements form parallel helices around a longitudinal axis of the catheter, wherein a distance separating the conductive elements is substantially constant between the constrained configuration and the helical configuration.

26. The neuromodulation catheter of claim 14 wherein the dielectric element is an elongated dielectric element disposed between conductive elements in the common portion of the length of the distal portion.

27. A method of performing neuromodulation within a target blood vessel of a human patient, the method comprising:
intravascularly delivering a neuromodulation catheter in a low-profile delivery configuration to a target treatment site within the target blood vessel, wherein the neuromodulation catheter comprises
at least two conductive elements that extend in parallel along a distal portion of the neuromodulation catheter;
a dielectric element separating the conductive elements; and
a sheath radially constraining the conductive and dielectric elements therein;
retracting the sheath from the conductive elements, thereby permitting the conductive and dielectric elements to assume a deployed configuration having a radially expanded, generally helical shape; and
selectively delivering energy to one or more of the conductive elements to modulate target nerves proximate to an inner wall of the target blood vessel.

28. The method of claim 27, further comprising:
advancing the sheath over the conductive and dielectric elements, thereby transforming the catheter into the low-profile delivery configuration; and
removing the neuromodulation catheter from the patient.

29. The method of claim 27 wherein selectively delivering energy comprises delivering bipolar electrical energy to the conductive elements.

30. The method of claim 27 wherein selectively delivering energy comprises delivering monopolar electrical energy to one or more of the conductive elements.

31. The method of claim 27 further comprising circulating a coolant fluid through hollow lumens in the conductive elements.

32. The method of claim 27 further comprising delivering an irrigating fluid through hollow lumens in the conductive elements, the conductive elements further including apertures that permit the irrigating fluid to be emitted from the conductive elements.

33. The method of claim 27 further comprising delivering an irrigating fluid through a hollow lumen in the dielectric material, the dielectric material further including apertures that permit the irrigating fluid to be emitted from the dielectric material.

34. The method of claim 27 wherein, when the conductive and dielectric elements assume the deployed configuration, the conductive elements are in apposition with the inner wall of the target blood vessel.
